(19) United States
(12) Patent Application Publication
(10) Pub. No.: US 2018/0021155 A1
(43) Pub. Date: Jan. 25, 2018

(54) TAPERED BODY AAA GRAFT

(71) Applicant: Cook Medical Technologies LLC, Bloomington, IN (US)

(72) Inventors: Rick Hadley, Otterbein, IN (US); Jarin A. Kratzberg, Lafayette, IN (US)

(73) Assignee: Cook Medical Technologies LLC, Bloomington, IN (US)

(21) Appl. No.: 15/655,126

(22) Filed: Jul. 20, 2017

Related U.S. Application Data
(60) Provisional application No. 62/365,103, filed on Jul. 21, 2016.

(51) Int. Cl. A61F 2/89; A61F 2/00; A61F 2/04

(52) U.S. Cl. CPC A61F 2/89; A61F 2/04; A61F 2/0031; A61F 2002/30092

(57) ABSTRACT

The present disclosure relates to an apparatus and method for deployment of a modular stent-graft system. In one embodiment, a modular system comprises a first stent graft having a tapered zone, a second stent graft configured for insertion within the first stent graft, the second stent graft comprising a tapered zone, wherein each tapered zone has an angle of taper and the angle of taper of the tapered zone of the second stent graft is substantially the same as the angle of taper of the tapered zone of the first stent graft, and wherein when the second stent graft is disposed within the first stent graft, the tapered zone of the second stent graft conforms precisely to the tapered zone of the first stent graft.

### RELATED APPLICATIONS

This application claims the benefit of priority of U.S. Provisional Application Ser. No. 62/365,103 filed Jul. 21, 2016, which us incorporated by reference in its entirety.

### TECHNICAL FIELD

The present invention relates to medical devices and more particularly to endovascular devices.

### BACKGROUND

Endoluminal prostheses may be inserted into a body lumen such as an anatomical vessel or duct for various purposes. Prostheses may maintain or restore patency in a formerly blocked or constricted passageway or they may be used for different procedures. For example, a prosthesis may include one or more stents disposed in or about a graft, and the stents may hold the graft in an open configuration to treat an aneurysm. Additionally, stents coupled to one or both ends of a graft may extend proximally or distally away from the graft to engage a healthy portion of a vessel wall away from a diseased portion of an aneurysm to provide endovascular graft fixation.

Modular stent graft pieces can be deployed in stages to form a combined stent graft assembly. First, a central or main stent graft body can be deployed. Subsequently, secondary or attachment stent graft body can be deployed and positioned within the main stent graft body.

One of the most common complications associated with endoluminal grafting for abdominal aortic aneurysms (“AAA”) is the stove piping effect that can occur in the overlap length into the proximal taper of a main stent graft body. It is desirable to create a stent graft assembly that reducing the stove piping effect and reduces the chance of turbulent flow of blood and possible thrombotic buildup through the main stent graft body.

### SUMMARY

According to a first aspect of the present disclosure, there is provided a modular stent graft system comprising a first stent graft having a first open end, a first cylindrical zone extending from the first open end for a first length, a tapered zone extending from the cylindrical zone for a second length, the tapered zone tapering from a first diameter to a second diameter smaller than the first diameter, and a second cylindrical zone extending from the tapered zone for a third length to a second open end, wherein the third length is greater than both the first length and the second length and the first length is greater than the second length; a second stent graft configured for insertion within the first stent graft, the second stent graft comprising a first open end, a tapered zone extending from the first open end for a first length, the tapered zone tapering from a first diameter to a second diameter smaller than the first diameter, and a cylindrical zone extending from the tapered zone to a second open end for a second length longer than the first length; wherein the first diameter of the tapered zone of the second stent graft is substantially the same as the first diameter of the tapered zone of the first stent graft, the second diameter of the tapered zone of the second stent graft is substantially the same as the second diameter of the tapered zone of the first stent graft, wherein each tapered zone has an angle of taper and the angle of taper of the tapered zone of the second stent graft is substantially the same as the angle of taper of the tapered zone of the first stent graft, wherein the second stent graft cylindrical zone is longer than the first stent graft second cylindrical zone such that when the second stent graft is disposed within the first stent graft and the tapered zones are mated, the second stent graft cylindrical zone extends beyond the second open end of the first stent graft, wherein the second stent graft comprises a first internal seal stent at the first open end of the second stent graft, the first seal stent having X proximal apices and a length, and a second internal seal stent directly adjacent and partially nesting with the first internal stent, the second seal stent having ½ X top apices and a length greater than the first seal stent; and wherein when the second stent graft is disposed within the first stent graft, the tapered zone of the second stent graft conforms precisely to the tapered zone of the first stent graft.

According to a second aspect of the present disclosure, there is provided a modular stent graft system comprising: a first stent graft having a first open end, a first cylindrical zone extending from the first open end for a first length, a tapered zone extending from the cylindrical zone for a second length, the tapered zone tapering from a first diameter to a second diameter smaller than the first diameter, and a second cylindrical zone extending from the tapered zone for a third length to a second open end, wherein the third length is greater than both the first length and the second length and the first length is greater than the second length; a second stent graft configured for insertion within the first stent graft, the second stent graft comprising a first open end, a tapered zone extending from the first open end for a first length, the tapered zone tapering from a first diameter to a second diameter smaller than the first diameter, and a cylindrical zone extending from the tapered zone to a second open end for a second length longer than the first length; wherein the first diameter of the tapered zone of the second stent graft is substantially the same as the first diameter of the tapered zone of the first stent graft, the second diameter of the tapered zone of the second stent graft is substantially the same as the second diameter of the tapered zone of the first stent graft; wherein each tapered zone has an angle of taper and the angle of taper of the tapered zone of the second stent graft is substantially the same as the angle of taper of the tapered zone of the first stent graft; and wherein the second stent graft cylindrical zone is longer than the first stent graft second cylindrical zone such that when the second stent graft is disposed within the first stent graft and the tapered zones are mated, the second stent graft cylindrical zone extends beyond the second open end of the first stent graft, and wherein when the second stent graft is disposed within the first stent graft, the tapered zone of the second stent graft conforms precisely to the tapered zone of the first stent graft.

According to a third aspect of the present disclosure, there is disclosed a method of deploying a modular stent graft system, the method comprising: inserting into the body a first stent graft having a first open end, a first cylindrical zone extending from the first open end for a first length, a tapered zone extending from the cylindrical zone for a second length, the tapered zone tapering from a first diameter to a second diameter smaller than the first diameter, and a second cylindrical zone extending from the tapered zone for a third length to a second open end, wherein the third length is greater than both the first length and the second length and the first length is greater than the second length; inserting into the body a second stent graft within the first stent graft, the second stent graft comprising a first open end, a tapered zone extending from the first open end for a first length, the tapered zone tapering from a first diameter to a second diameter smaller than the first diameter, and a cylindrical zone extending from the tapered zone to a second open end for a second length longer than the first length; wherein the first diameter of the tapered zone of the second stent graft is substantially the same as the first diameter of the tapered zone of the first stent graft, the second diameter of the tapered zone of the second stent graft is substantially the same as the second diameter of the tapered zone of the first stent graft, wherein each tapered zone has an angle of taper and the angle of taper of the tapered zone of the second stent graft is substantially the same as the angle of taper of the tapered zone of the first stent graft, wherein the second stent graft cylindrical zone is longer than the first stent graft second cylindrical zone such that when the second stent graft is disposed within the first stent graft and the tapered zones are mated, the second stent graft cylindrical zone extends beyond the second open end of the first stent graft, wherein when the second stent graft is disposed within the first stent graft, the tapered zone of the second stent graft conforms precisely to the tapered zone of the first stent graft.

### BRIEF DESCRIPTIONS OF THE DRAWINGS

An embodiment of the present invention will not be described by way of example with reference to the accompanying drawings, in which:

FIG. 1 illustrates one example of a main stent graft body or device.

FIG. 2 illustrates one example of an attachment stent graft having a flared proximal end.

FIG. 3 illustrates the proximal end of the attachment stent graft shown in FIG. 2.

FIG. 4 illustrates one example of a main stent graft body coupled to an attachment stent graft at a tapered mating area.

FIG. 5 illustrates one example of a main stent graft body coupled to an attachment stent graft at a distal mating area.

FIG. 6 illustrates a detailed view of the tapered mating area shown in FIG. 4.

FIG. 7 illustrates a perspective view of the assembly of FIG. 6.

FIG. 8 illustrates a detailed view of the distal mating area shown in FIG. 5.

FIG. 9 illustrates a perspective view of the assembly of FIG. 8.

FIG. 10 illustrates the flow of blood through a known stent graft assembly.

FIG. 11 illustrates the flow of blood through a stent graft assembly.

FIG. 12 illustrates the flow of blood through a known stent graft assembly.

FIG. 13 illustrates the flow of blood through a stent graft assembly.

### DETAILED DESCRIPTION

In the present application, the term “proximal” when referring to a delivery device refers to a direction that is farthest away from the operator using a delivery device, while the term “distal” refers to a direction that is generally closest to the operator using the delivery device. The proximal and distal ends of a delivery device can also be referred to as the introduction end of the delivery device and the operator end of the delivery device. The operator end of the delivery device is that portion of the device that is intended to remain outside of a patient during a procedure. When referring to the prosthesis itself relative to the delivery device, the proximal end of the prosthesis is that part of the prosthesis nearest the delivery end of the delivery device and the distal end of the prosthesis is that end that is closest to the operator end of the delivery device. When referring to the prosthesis relative to placement in the human body, the ends of the various devices and parts of devices may be referred to as the inflow end (that end that receives fluid first, and the outflow end (that end from which the fluid exits).

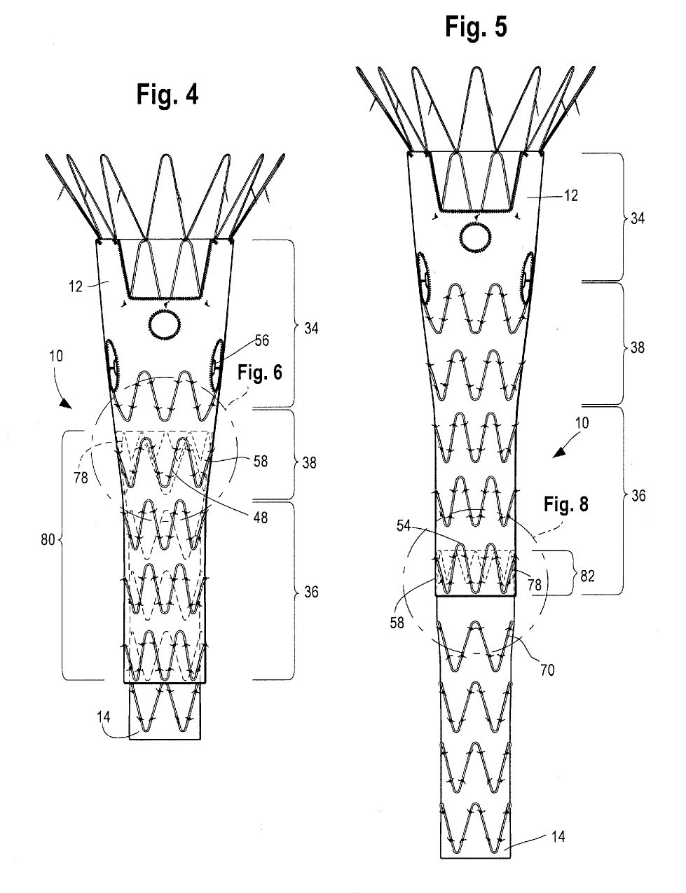

The stent graft assembly 10 of the present invention (shown for example in FIGS. 4-5) may include a main stent graft body or device 12 and a second graft body or attachment stent graft 14. In one example, the main stent graft body 12 may be a Cook Medical Zenith Pivoting Branch Graft showing pivoting fenestrations as disclosed in U.S. Pat. Nos. 8,702,786, 8,795,349, 9,351,822, 8,870,939 and US Patent Publication No. 2014/0277335, incorporated herein in their entirety. In one example, the main stent graft body 12 or attachment stent graft 14 may be a fenestrated graft such as those shown in U.S. Pat. Nos. 9,072,621, 9,072,621, 8,523,934, 9,060,887, 8,172,895, 7,833,259, 7,413,573, 9,149,382, and US Publication Nos. 2015-0112420, 2016-0022411, incorporated herein in their entirety. In one example, the attachment stent graft 14 may be a Cook Medical Zenith AAA Stent Graft.

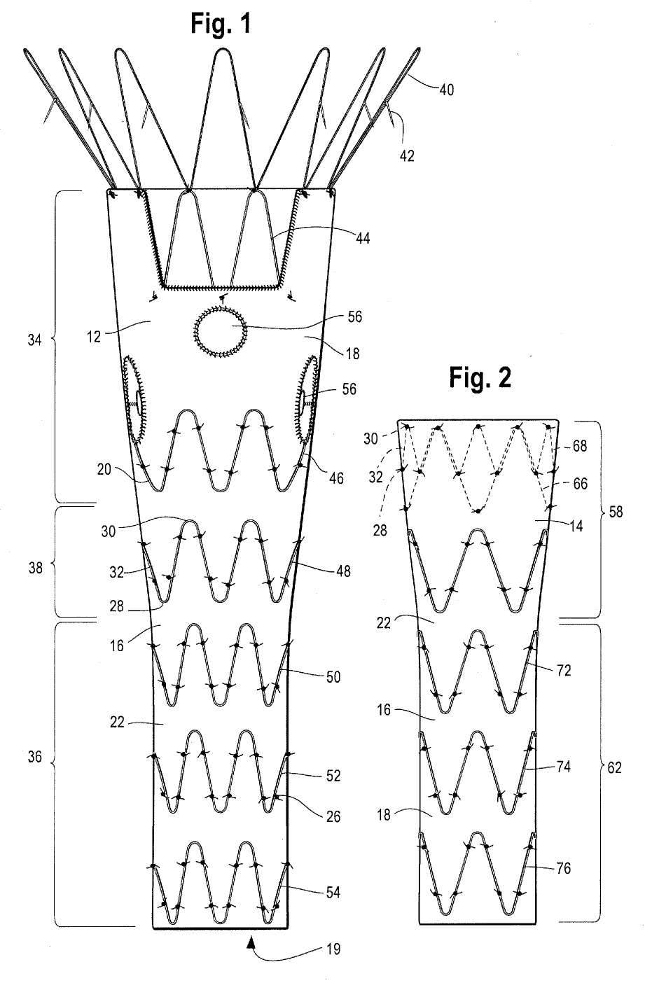

The main stent graft body 12 and attachment stent graft 14 will now be described in greater detail. FIG. 1 illustrates one example of a main stent graft body or device 12. FIG. 2 illustrates one example of an attachment stent graft 14 having a flared proximal end. In this embodiment, the main stent graft body 12 and attachment stent graft 14 may comprise a tubular body 16 of a biocompatible graft material 18 with a lumen 19 running therethrough.

The main stent graft body 12 and attachment stent graft 14 may be supported with one or more stents 20 that are secured to and along the graft material 18 either along the outer surface 22 or inner surface 24 of the graft material 18 such as by sutures 26. In one example, stent 20 may be a Z-stent. For example, stent 20 may have a distal end with a series of distal apices 28 and a proximal end with a series of proximal apices 30. Stent 20 may also have one or more elongate struts 32 connecting the distal apices 28 to the proximal apices 30.

Suitable stents 20 for use in connection with the main stent graft body 12 or the attachment stent graft 14 described herein may be self-expanding or mechanically-expandable stents or both, and may be deployed according to conventional methodology. A self-expanding stent may be manufactured from a shape-memory alloy, such as nickel titanium alloy (Nitinol). If the stent comprises a self-expanding material such as Nitinol, the stent may be heat-set into the desired expanded state whereby the stent can assume a relaxed radially expanded configuration. The stent may be made from other metals and alloys that allow the stent to return to its original expanded configuration upon deployment, such as, for example, stainless steel, cobalt-chrome alloys, amorphous metals, and/or non-metallic materials as would be recognized by one of skill in the art. Additionally or alternatively, the main stent graft body 12 and the attachment stent graft 14 may be mechanically expanded, such as through the use of an expandable balloon placed within the lumen 19 of the stent graft and then radially outwardly expanded.

The main stent graft body 12 may have a proximal end portion 34 and a distal end portion 36, with a tapered transition portion 38 that interconnects the proximal end portion 34 and the distal end portion 36. The distal end portion 36 may have a constant diameter and the proximal end portion 34 may have a selected larger diameter. An attachment stent 40 may be secured to the proximal end portion 34, with the attachment stent 40 extending proximally from the graft material 18. The attachment stent 40 may and have one or more barbs 42 configured to secure the main stent graft body 12 to a vessel wall.

The main stent graft body 12 may have several additional stents 44, 46, 48, 50, 52 and 54 adjacent to the attachment stent 40. In one example, stent 44 may be secured to the inner surface 24 of the graft material 18 in the proximal end portion 34 of the main stent graft body 12. Stents 46, 48, 50, 52, and 54 may be secured about the outer surface 22 of the graft material 18 along the length thereof. In one example, stents 46, 48, 50, 52, and 54 are located distal to the attachment stent 40 and stent 44.

The proximal end portion 34 may contain a plurality of radiopaque markers (not shown) such as gold marker members for facilitating fluoroscopic visualization of the proximal end of the graft material 18. For example, radiopaque markers may be used to place the main body stent graft 12 distal to the renal arteries (not shown).

As shown in FIG. 1, one or more fenestrations 56 may be placed on the biocompatible graft material 18. These fenestrations 56 may substantially align with the expected position of a branch vessel in the patient. There is, however, some variation both circumferentially and longitudinally in the position of the vessels.

FIG. 2 illustrates one example of an attachment stent graft 14 having a flared proximal end 58. The attachment stent graft 14 may be configured to pair with the main stent graft body 12. In one example, the main stent graft body 12 is configured to receive the attachment stent graft 14 as described in greater detail below.

The attachment stent graft 14 shown in FIG. 2 may have a flared proximal end portion 58 and a distal end portion 62. The distal end portion 62 may have a constant diameter and the flared proximal end portion 58 may have a selected larger diameter. The distal end portion 62 may be a unibody tube (as shown), or it may be a bifurcated stent graft having a body portion and two or more legs. In one example, the bifurcated stent graft comprises a shorter leg and a longer leg.

The attachment stent graft 14 may have several stents 66, 68, 70, 72, 74, and 76 which may be secured to the graft material 18 along the length of the attachment stent graft 14. In one example, a first stent 66 and a second stent 68 may be secured to the inner surface 24 of the graft material 18 near the flared proximal end portion 58. In one example, additional stents 70, 72, 74, and 76 may be secured about the outer surface 22 of the graft material 18.

Importantly, the proximal end of the attachment stent graft 14 may have a flared proximal end portion 58. In one example, the flared portion 58 may complement or conform to the transition portion 36 on the interior of the main stent graft body 12. For example, as seen comparing FIGS. 1 and 2 side by side, the flared proximal end 58 of the attachment stent graft 14 and the transition portion 36 of the main stent graft body 12 are similar in shape. The complementary flared proximal end 58 of the attachment stent graft 14 and the transition portion 36 of the main stent graft body 12 may allow a better mating area between the main stent graft body 12 and the attachment stent graft 14 (as described below and shown in FIGS. 4-9).

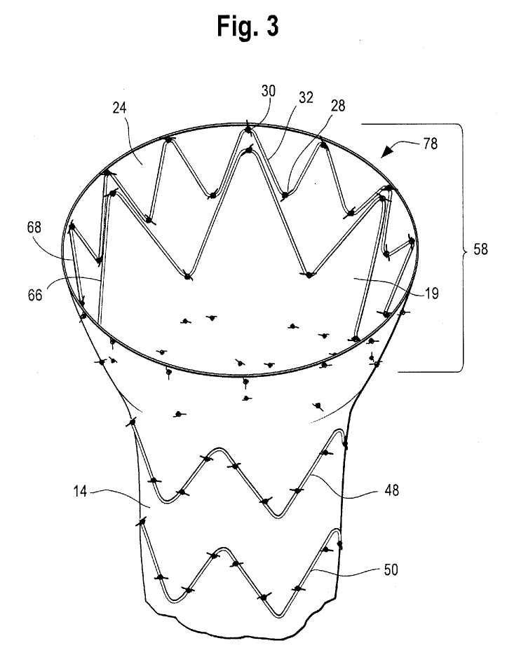

FIG. 3 illustrates the proximal end of the attachment stent graft shown in FIG. 2. In one example, the flared proximal end portion 58 of the attachment stent graft 14 may have a stacked stent arrangement 78. The stacked stent arrangement 78 may be located in the flared proximal end 58 of the attachment stent graft 14. The stacked stent arrangement 78 may allow for a tighter seal when the attachment stent graft 14 is placed into a lumen 19 in the main stent graft body 12.

In one example of a stacked stent arrangement 78, a first stent 66 and a second stent 68 are mounted on an interior surface 24 of the graft material 18 in the attachment stent graft 14. The first stent 66 and second stent 68 may be formed as a wire ring that has proximal and distal generally curved apex portions (apices) 30, 28 separated from each other by intermediate generally straight portions called struts 32.

First stent 66 and second stent 68 may not have the same proximal to distal length. For example, as illustrated in FIG. 3 the second stent 68 has a shorter proximal-distal length than the first stent 66. In another embodiment (not shown), the first stent 66 and the second stent 68 may have the same proximal to distal length.

In one embodiment the first stent 66 and second stent 68 may overlap axially such that the stents apices of the second stent 68 nest within the apices of the first stent 66. In another embodiment the first stent 66 and second stent 68 do not overlap or nest. For example, the first stent 66 and second stent 68 do not overlap axially, but are very close together. In one example, the first stent 66 is located slightly distal to the second stent 68.

In one embodiment (not shown), each of the proximal apices 30 of the first stent 66 may be circumferentially offset from each of the proximal apices 30 of the second stent 68. In another example, each of the distal apices 30 of the first stent 66 may be circumferentially offset from each of the distal apices 30 of the second stent 68. In another example, the first stent 66 and the second stent 68 comprise identical geometries. The two stents 66 and 68 may be arranged in an out-of-phase alignment or may be in-phase alignment. The first stent 66 and second stent 68 may have any configuration and geometry as disclosed in U.S. Pat. No. 8,728,145, which is incorporated by reference herein.

The attachment stent graft 14 and the main stent graft body 12 may be coupled or connected. FIGS. 4 and 5 illustrate two examples of a main stent graft body 12 coupled to an attachment stent graft 14.

In one example, the attachment stent graft 14 may have a reduced diameter delivery configuration (not shown) and a deployment configuration. When in the delivery configuration, the attachment stent graft 14 can be deployed into a lumen 19 in the main stent graft body 12.

The attachment stent graft 14 can be positioned and deployed to any location along the length of the main stent graft body 12. In this way, the stent graft assembly 10 may be any suitable customizable length. For example, if the attachment stent graft 14 is placed in a more proximal location of the main stent graft 12, the total length of the stent graft assembly 10 may be shorter. If the attachment stent graft 14 is placed in a more distal location on the main stent graft 12, the total length of the stent graft assembly 10 may be longer.

When in a deployed state, the attachment stent graft 14 may expand and conform to the shape of the interior of the main stent graft body 12.

FIG. 4 illustrates one example of a main stent graft body 12 coupled to an attachment stent graft 14 at a tapered mating area 80. As illustrated in FIG. 4, the flared proximal end 58 of the attachment stent graft 14 may be coupled to the main stent graft body 12 at a tapered mating area 80. The tapered mating area 80 may be located in the area just distal of fenestrations 56. A portion of the tapered mating area 80 may be in the tapered transition portion 38 that interconnects the proximal end portion 34 and the distal end portion 36 of the main stent graft body 12. The flared proximal end 58 of the attachment stent graft 14 may conform to the shape of the tapered transition portion 38 of the main stent graft body 12.

The radial force of the stacked stent arrangement 78 may hold the attachment stent graft 14 tightly against the interior of the main stent graft body 12. In this way, there is a tight seal between the attachment stent graft 14 and the main stent graft body 12. As described in more detail below, this tight seal can prevent turbulent vascular flow through the lumen 19 of the stent graft assembly 10.

FIG. 5 illustrates one example of a main stent graft body 12 coupled to an attachment stent graft 14 at a distal mating area 82. As illustrated in FIG. 5, the flared proximal end 58 of the attachment stent graft 14 may be coupled to the main stent graft body 12 at a distal mating area 82. The distal mating area 82 may be in the distal end portion 36 of the main stent graft body.

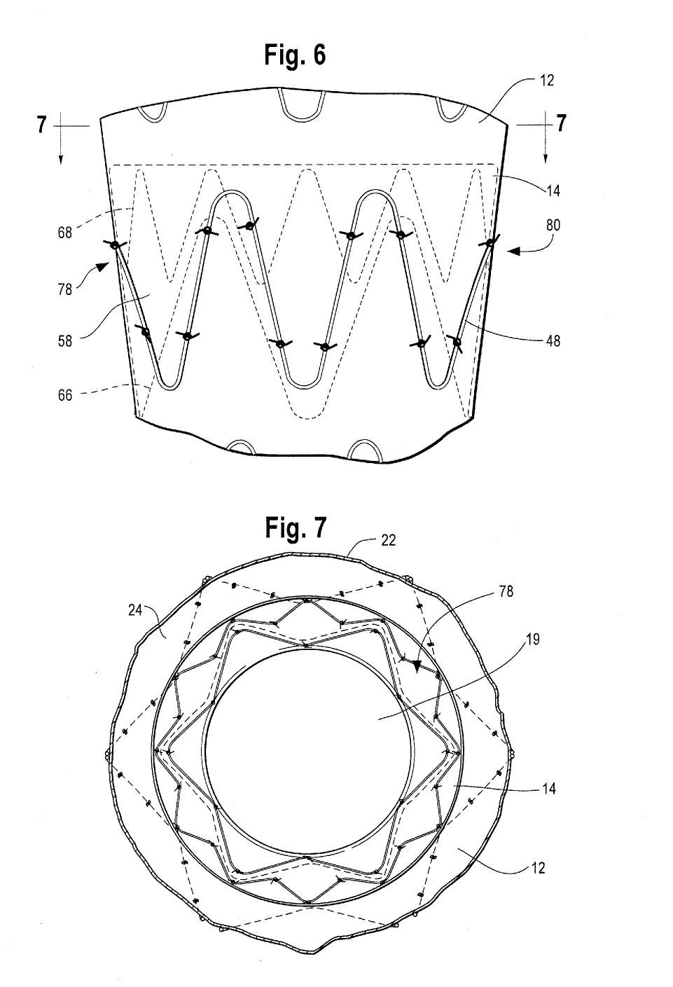

FIG. 6 illustrates a close up of the tapered mating area 80 shown in FIG. 4. The attachment stent graft 14 may be disposed in the lumen 19 of the main stent graft body 12. In this example, the flared proximal end 58 of the attachment stent graft 14 is disposed in the main stent graft body 12 in the tapered mating area 80. The stacked stent arrangement 78 mounted on the interior graft 24 in the flared proximal end 58 of the attachment stent graft 14 may be at the same longitudinal level as stent 48 on the main stent graft body 12.

FIG. 7 illustrates a perspective view of the assembly of FIG. 6. The main stent graft body 12 may have a lumen 19 extending therethrough. The attachment stent graft 14 may be disposed in the lumen 19 of the main stent graft body 12. A stacked stent arrangement 78 may be disposed on the flared proximal end 58 of the attachment stent graft 14.

As shown in FIG. 7, the shape of the flared proximal end 58 of the attachment stent graft 14 and the tapered transition portion 38 of the main stent graft body 12 are complimentary such that a tight seal can be created between the main stent graft body 12 and the attachment stent graft 14. As will be described in greater detail below, a tight seal can prevent turbulent vascular flow through the lumen 19 of the assembly 10.

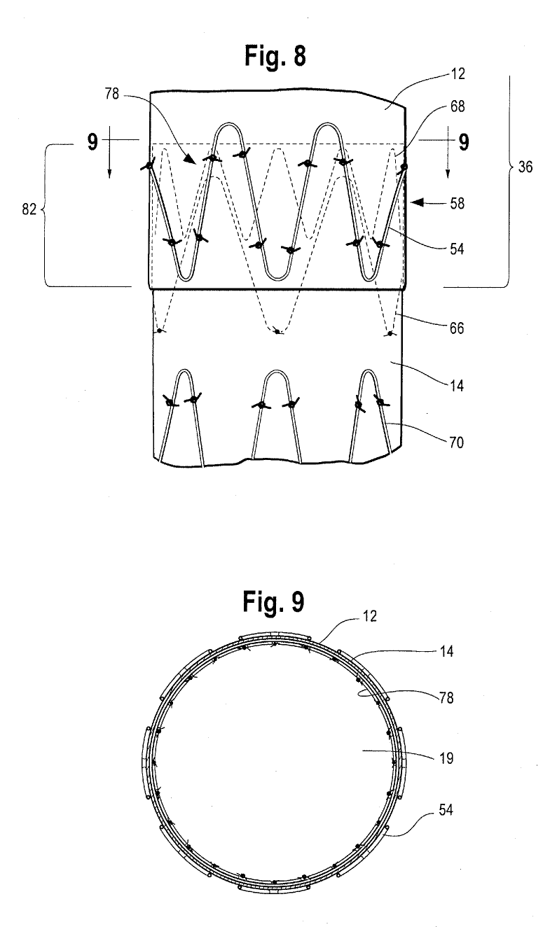

FIG. 8 illustrates a detailed view of the distal mating area 82 illustrated in FIG. 5. The attachment stent graft 14 may be disposed in the lumen 19 of the main stent graft body 12. In this example, the flared proximal end 58 of the attachment stent graft 14 is disposed in the main stent graft body 12 in the distal mating area 82.

As shown in FIG. 8, a stent 54 on the distal portion 36 of the main stent graft body 12 may be located on the outer surface of the main stent graft body 12. The attachment stent graft 14 may have a stacked stent arrangement 78 mounted on an interior surface 24 of the flared proximal end 58 of the attachment stent graft 14. The stacked stent arrangement 78 may be at the same longitudinal level as a stent 54 on the distal portion 36 of the main stent graft body 12.

FIG. 9 illustrates a perspective view of the assembly of FIG. 8. The main stent graft body 12 may have a lumen 19 extending therethrough. The attachment stent graft 14 may be disposed in the lumen 19 of the main stent graft body 12. A stacked stent arrangement 78 may be disposed on the flared proximal end 58 of the attachment stent graft 14. In this example, the attachment stent graft 14 is disposed at the distal end portion 36 of the main stent graft body 12.

As shown in FIG. 9, a tight seal can be created between the main stent graft body 12 and the attachment stent graft 14. As will be described in greater detail below, a tight seal can prevent turbulent vascular flow through the lumen 19 of the stent graft assembly 10.

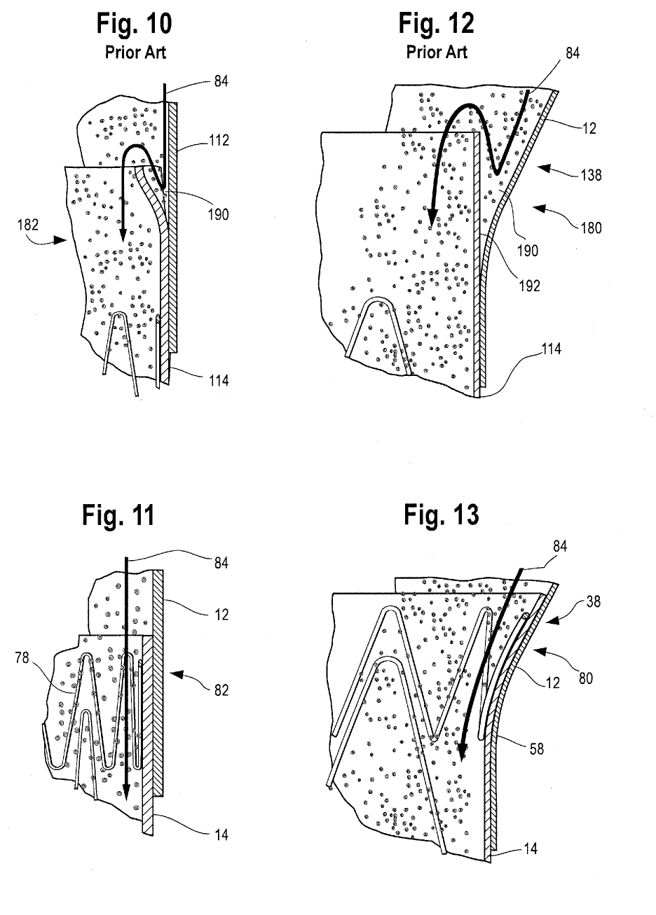

FIG. 10 illustrates the flow of blood through a known stent graft assembly 110 where a main stent graft body 112 is coupled to a conventional attachment stent graft 114 at a distal mating area 182. As shown in FIG. 10, blood flow 84 can travel in a proximal 86 to distal 88 direction through the lumen 119 of the main stent graft body 112. A conventional attachment graft 114 may be disposed in the lumen 119 of the main stent graft body 112. The seal between the conventional attachment graft 114 and the main stent graft body 112 may not be very tight and stove piping may occur. When stove piping occurs, the blood flow 84 may be turbulent at the transition between the main stent graft body 112 and the conventional attachment graft 114. As shown in FIG. 10, the blood flow may not take a straight path through the lumen of the stent graft assembly 10 but rather may get caught in a gap 190 in a distal mating area 182.

FIG. 11 illustrates the flow of blood 84 through a stent graft assembly 10 of the present disclosure where a main stent graft body 12 is coupled to an attachment stent graft 14 at a distal mating area 82 wherein the proximal end of the attachment stent graft has a stacked stent arrangement. As shown in FIG. 11, a tight seal created by the stacked stent arrangement 78 in the attachment stent graft 14 can allow for blood to flow in a relatively straight path in a proximal 86 to distal 88 direction. The tighter seal can reduce the stove piping effect that can be seen with conventional devices. It can also reduce the risk of turbulent blood flow and also reduces the risk for thrombolytic build-up at a seal area 82. The tighter seal created by the stacked stent arrangement 78 can also allow a constant seal. The stacked stent arrangement 78 at the proximal end of the attachment stent graft 14 can be flexible and adjustable and allow for deep connection delivery of grafts.

FIG. 12 illustrates the flow of blood through a known stent graft assembly 10 where a main stent graft body is coupled to an attachment stent graft at a tapered mating area. As shown in FIG. 12, a conventional attachment stent graft 14 may have a straight proximal end 192 (as opposed to the tapered proximal end shown in this disclosure at FIGS. 2-4). As a result, a gap 190 may occur between the tapered transition area 138 on the main stent graft body 12 and the proximal end 192 of the conventional attachment stent graft 14. Blood flowing through the assembly 10 may not take a straight path through the lumen of the stent graft assembly 10 but rather the blood may get caught in a gap 190 in a seal area 180. This gap can create a turbulent blood flow.

FIG. 13 illustrates the flow of blood through a stent graft assembly 10 where a main stent graft body 12 is coupled to an attachment stent graft 14 at a tapered mating area 80 wherein the flared proximal end 58 of the attachment stent graft 14 is tapered and has a stacked stent arrangement. In this arrangement, the tapered proximal portion 58 of the attachment stent graft 14 may create a tight seal with the tapered transition portion 38 of the main stent graft body 12.

As shown in FIG. 13, the tighter seal created by the stacked stent arrangement 78 and the tapered proximal portion 58 of the attachment stent graft 14 can allow for blood to flow in a relatively straight path in a proximal 86 to distal 88 direction. The tighter seal can reduce the stove piping effect that can be seen with conventional devices. It can also reduce the risk of turbulent blood flow and also reduces the risk for thrombolytic build-up at a seal area 80. The tighter seal created by the stacked stent arrangement 78 and tapered transition portion 58 can also allow a constant seal. The stacked stent arrangement 78 and the tapered proximal portion 58 of the attachment stent graft 14 can be flexible and adjustable and allow for deep connection delivery of grafts.

While various embodiments of the invention have been described, the invention is not to be restricted except in light of the attached claims and their equivalents.

We claim:

1. A modular stent graft system comprising:
a first stent graft having a first open end, a first cylindrical zone extending from the first open end for a first length, a tapered zone extending from the cylindrical zone for a second length, the tapered zone tapering from a first diameter to a second diameter smaller than the first diameter, and a second cylindrical zone extending from the tapered zone for a third length to a second open end, wherein the third length is greater than both the first length and the second length and the first length is greater than the second length;
a second stent graft configured for insertion within the first stent graft, the second stent graft comprising a first open end, a tapered zone extending from the first open end for a first length, the tapered zone tapering from a first diameter to a second diameter smaller than the first diameter, and a cylindrical zone extending from the tapered zone to a second open end for a second length longer than the first length;
wherein the first diameter of the tapered zone of the second stent graft is greater than or equal to the first diameter of the tapered zone of the first stent graft, the second diameter of the tapered zone of the second stent graft is greater than or equal to the second diameter of the tapered zone of the first stent graft;
wherein each tapered zone has an angle of taper and the angle of taper of the tapered zone of the second stent graft is the same as the angle of taper of the tapered zone of the first stent graft;
wherein the second stent graft cylindrical zone is longer than the first stent graft second cylindrical zone such that when the second stent graft is disposed within the first stent graft and the tapered zones are mated, the second stent graft cylindrical zone extends beyond the second open end of the first stent graft;
wherein the second stent graft comprises a first internal seal stent at the first open end of the second stent graft, the first seal stent having X proximal apices and a length, and a second internal seal stent directly adjacent and partially nesting with the first internal stent, the second seal stent having ½ X top apices and a length greater than the first seal stent; and
wherein when the second stent graft is disposed within the first stent graft, the tapered zone of the second stent graft conforms precisely to the tapered zone of the first stent graft.

2. The modular stent graft system of claim 1, wherein the first stent graft has an interior and exterior and the second stent graft has an interior and exterior, wherein when the second stent graft is disposed within the first stent graft, a portion of the interior of the first stent graft directly abuts a portion of the exterior of the second stent graft in graft to graft opposition without any stents in between.

3. The modular stent graft system of claim 1, wherein the first stent graft's tapered zone has the same dimensions as the second stent graft's tapered zone.

4. The modular stent graft system of claim 1, wherein when the second stent graft is disposed within the first stent graft, the tapered zone of the second stent graft creates a seal with the tapered zone of the first stent graft.

5. The modular stent graft system of claim 1, wherein when the second stent graft comprises at least two fenestrations, and wherein, when the second stent graft is disposed within the first stent graft, the first open end of the second stent graft lies below the at least two fenestrations.

6. The modular stent graft system of claim 1, wherein the first seal stent and the second seal stent are in phase and every other top apex of the first seal stent nests with an apex of the second seal stent.

7. The modular stent graft system of claim 6, wherein the top apices of the first seal stent abut an edge of the first stent graft at the first open end.

8. The modular stent graft system of claim 1, wherein the second stent graft is a bifurcated grafting including a body portion and two leg portions extending from the body portion.

9. The modular stent graft system of claim 7, wherein the first stent graft comprises a stent disposed about the exterior of the tapered zone of the first stent graft and having top apices, and wherein, when the second stent graft is disposed in the first stent graft, the stent disposed about the exterior of the tapered zone of the first stent graft are in phase with the second seal stent.

10. The modular stent graft of claim 1, wherein the first stent graft comprises a stent disposed about the exterior of the tapered zone of the first stent graft and has top apices and bottom apices, wherein the circumference of the stent at the top apices is greater than the circumference of the stent at the bottom apices such that the stent tapers from the top apices to the bottom apices.

11. The modular stent graft of claim 10, wherein the second seal stent has top apices and bottom apices, wherein the circumference of the stent at the top apices is greater than the circumference of the stent at the bottom apices such that the stent tapers from the top apices to the bottom apices.

12. A modular stent graft system comprising:
a first stent graft having a first open end, a first cylindrical zone extending from the first open end for a first length, a tapered zone extending from the cylindrical zone for a second length, the tapered zone tapering from a first diameter to a second diameter smaller than the first diameter, and a second cylindrical zone extending from the tapered zone for a third length to a second open end, wherein the third length is greater than both the first length and the second length and the first length is greater than the second length;
a second stent graft configured for insertion within the first stent graft, the second stent graft comprising a first open end, a tapered zone extending from the first open end for a first length, the tapered zone tapering from a first diameter to a second diameter smaller than the first diameter, and a cylindrical zone extending from the tapered zone to a second open end for a second length longer than the first length;
wherein the first diameter of the tapered zone of the second stent graft is the same as the first diameter of the tapered zone of the first stent graft, the second diameter of the tapered zone of the second stent graft is the same as the second diameter of the tapered zone of the first stent graft;
wherein each tapered zone has an angle of taper and the angle of taper of the tapered zone of the second stent graft is the same as the angle of taper of the tapered zone of the first stent graft;
wherein the second stent graft cylindrical zone is longer than the first stent graft second cylindrical zone such that when the second stent graft is disposed within the first stent graft and the tapered zones are mated, the second stent graft cylindrical zone extends beyond the second open end of the first stent graft, and
wherein when the second stent graft is disposed within the first stent graft, the tapered zone of the second stent graft conforms precisely to the tapered zone of the first stent graft in graft to graft apposition with no stent in between.

13. The modular stent graft system of claim 12, wherein the first stent graft has an interior and an exterior and the second stent graft has an interior and an exterior, wherein when the second stent graft is disposed within the first stent graft, a portion of the inside of the first stent graft directly abuts a portion of the outside of the second stent graft to form a fluid tight seal between the exterior of the second stent graft and the interior of the first stent graft at each of the respective tapered zones.

14. The modular stent graft system of claim 12, wherein the first stent graft's tapered zone has the same dimensions as the second stent graft's tapered zone.

15. The modular stent graft system of claim 12, wherein the first seal stent and the second seal stent are in phase and every other top apex of the first seal stent nests with an apex of the second seal stent.

16. The modular stent graft system of claim 15, wherein the top apices of the first seal stent abut an edge of the first stent graft at the first open end.

17. The modular stent graft system of claim 12, wherein the first stent graft comprises a stent disposed about the exterior of the tapered zone of the first stent graft and having top apices, and wherein, when the second stent graft is disposed in the first stent graft, the stent disposed about the exterior of the tapered zone of the first stent graft are in phase with the second seal stent.

18. The modular stent graft of claim 16, wherein the first stent graft comprises a stent disposed about the exterior of the tapered zone of the first stent graft and has top apices and bottom apices, wherein the circumference of the stent at the top apices is greater than the circumference of the stent at the bottom apices such that the stent tapers from the top apices to the bottom apices.

19. A method of deploying a modular stent graft system, the method comprising:
inserting into the body a first stent graft having a first open end, a first cylindrical zone extending from the first open end for a first length, a tapered zone extending from the cylindrical zone for a second length, the tapered zone tapering from a first diameter to a second diameter smaller than the first diameter, and a second cylindrical zone extending from the tapered zone for a third length to a second open end, wherein the third length is greater than both the first length and the second length and the first length is greater than the second length;
inserting into the body a second stent graft within the first stent graft, the second stent graft comprising a first open end, a tapered zone extending from the first open end for a first length, the tapered zone tapering from a first diameter to a second diameter smaller than the first diameter, and a cylindrical zone extending from the tapered zone to a second open end for a second length longer than the first length; wherein the first diameter of the tapered zone of the second stent graft is the same as the first diameter of the tapered zone of the first stent graft, the second diameter of the tapered zone of the second stent graft is the same as the second diameter of the tapered zone of the first stent graft, wherein each tapered zone has an angle of taper and the angle of taper of the tapered zone of the second stent graft is the same as the angle of taper of the tapered zone of the first stent graft, wherein the second stent graft cylindrical zone is longer than the first stent graft second cylindrical zone such that when the second stent graft is disposed within the first stent graft and the tapered zones are mated, the second stent graft cylindrical zone extends beyond the second open end of the first stent graft, wherein when the second stent graft is disposed within the first stent graft, the tapered zone of the second stent graft conforms precisely to the tapered zone of the first stent graft to create a fluid tight seal between graft material between the tapered zones without any stent in between.

20. The method of claim 19, wherein the second stent graft's tapered zone is deployed within the first stent graft's tapered zone.
